(19) United States
(12) Patent Application Publication
(10) Pub. No.: US 2014/0023701 A1
(43) Pub. Date: Jan. 23, 2014

(54) Method and Composition for Ameliorating the Effects tor a Subject Exposed to Radiation or Other Sources of Oxidative Stress

(71) Applicants: SUPPLEMENT RESEARCH AND DEVELOPMENT LLC, Houston, TX (US); Nugevity LLC, Pleasant Hill, CA (US)

(72) Inventors: Carlos A. Montesinos, Katy, TX (US); Jeffrey A. Jones, Houston, TX (US)

(73) Assignees: NUGEVITY LLC, Pleasant Hill, CA (US); SUPPLEMENT RESEARCH AND DEVELOPMENT LLC, Houston, TX (US)

(21) Appl. No.: 14/012,990

(22) Filed: Aug. 28, 2013

Related U.S. Application Data
(63) Continuation-in-part of application No. 13/439,546, filed on Apr. 4, 2012.
(60) Provisional application No. 61/473,057, filed on Apr. 7, 2011.
(60) Provisional application No. 61/489,631, filed on May 24, 2011.

(51) Int. Cl. A61K 36/88; A61K 31/385; A61K 31/07; A61K 31/7048; A61K 31/56; A61K 31/352; A61K 36/31; A61K 36/82; A61K 38/44; A61K 31/122

(52) U.S. Cl. CPC A61K 36/88; A61K 38/446; A61K 31/385; A61K 31/07; A61K 31/122; A61K 31/56; A61K 31/352; A61K 36/31; A61K 36/82; A61K 31/7048
USPC 424/450; 424/638; 424/639

(57) ABSTRACT

Radiation-oxidative exposure treatment compositions comprise a mixture of micronutrient multivitamin and trace elements, a mixture of antioxidants and chemopreventative agents, and optionally a mixture of fatty acids. Micronutrient multivitamin and trace elements mixtures include vitamins A, Bp, B1, B2, B3, B5, B6, B7, B9, B12, C, D, E and K; inositol; calcium, iodine, magnesium, zinc, selenium, copper, manganese, chromium, molybdenum, potassium, boron and vanadium. Mixtures of non-essential antioxidants and chemopreventative agents include bioflavins, alpha lipoic acid, N-acetyl-L-cysteine (optionally) lutein, lycopene, astaxanthin, plant sterols, isoflavones, garlic extract, which provides allicin; green tea extract, cruciferous vegetable extract, fruit extracts, coenzyme Q-10, and resveratrol. Fatty acid mixtures include eicosapentaenoic acid and docosahexaenoic acid.

Methods of treatment of a subject exposed to a radiation source or an oxidative stress with the radiation-oxidative exposure treatment composition include the step of administering to the subject a daily dose of the radiation-oxidative exposure treatment composition such that the life shortening effects induced by the radiation source or the oxidative stress are ameliorated.

### CROSS-REFERENCE TO RELATED APPLICATIONS

This application is a continuation-in-part of copending U.S. Nonprovisional patent application Ser. No. 13/439,546 filed Apr. 4, 2012, Confirmation No. 8360, which claims priority from U.S. Provisional Application No. 61/473,057 filed Apr. 7, 2011, and U.S. Provisional Application No. 61/489,631, filed May 24, 2011. For purposes of United States patent practice, this application incorporates the contents of these applications by reference in their entirety.

### STATEMENT REGARDING FEDERALLY SPONSORED RESEARCH OR DEVELOPMENT

Pursuant to an assignment to Jeffrey A. Jones, a former U.S. Government Employee, the U.S. Government retains, pursuant to 15 U.S.C. 3710d(a), a nonexclusive, nontransferable, irrevocable, paid-up license to practice the invention in the above-referenced related applications or have the invention in the above-references related applications practiced throughout the world by or on behalf of the U.S. Government.

### BACKGROUND OF THE INVENTION

1. Field of the Invention

The field of invention relates to compositions and methods useful for pre-treating and treating a subject exposed to radiation. More specifically, the field of invention relates to compositions and methods for reducing the risk for and ameliorating the radiation-induced life shortening effects from exposure to a radiation source.

2. Description of the Related Art

Oxidative damage is the result of the human body metabolizing oxygen so that the cells can produce the energy that runs all the chemical reactions that sustain life. During this critical process, the body produces dangerous molecules that react with cell proteins and DNA to cause irreversible damage.

Oxidative damage is well documented during many activities, including space flight, lunar exploration and space walks. Exposure to oxidative insults occurs to astronauts during extravehicular activities (EVA), including increased oxygen exposure (hyperoxia), radiation, and exercise. Risks from increased oxidative damage include increased muscle fatigue, increased risk for cataracts, macular degeneration, cardiovascular disease, and many forms of cancer, as well as a number of other chronic diseases. Currently there is no effective countermeasure to mitigate oxidative damage during these activities. As humanity contemplates lunar missions, with greatly increased EVA frequency and durations, mitigating oxygen-related health risks is important.

Ionizing radiation induces nuclear DNA strand breaks, which initiate a transfer to the mitochondria of both pro-apoptotic and anti-apoptotic molecules. The molecular events that occur early in the initiation of apoptosis originate at the mitochondrial membrane. The events include molecular sequelae of both oxidative and nitrosative stress, which produces rapid depletion of antioxidant stores. Antioxidant depletion at the mitochondria associates with disruption of cytochrome C binding to cardiolipin, mitochondrial membrane disruption, and leakage into the cytoplasm of cytochrome C. These disruptions and ruptures initiate a cascade of molecular events that eventually lead to apoptosis.

There are many sources of oxidative stress in the lives of workers, whether they work in nuclear power facilities, on the front lines of international conflicts, in hospitals, or in the reaches of outer space. The exposure dose can vary substantially, but at minimum will accelerate the aging of their organ systems, and at worse could result in acute exposure syndromes that may be fatal. A common thread of the oxidative stress exposures is reactive oxygen species (ROS)-binding to critical cellular organelles and molecules, which can result in cellular dysfunction, mutation of nucleic acids, or even apoptotic cell death. Currently there are no proven countermeasures for these exposures, aside from a clinical agent, amifostine, which reduces mucositis and other side effects from radiation therapy dose in cancer patients, and Iodine in the form of potassium iodide tablets, which reduces the likelihood of thyroid exposure to radioactive iodine.

Radiological terrorism, nuclear accidents, and astronauts outside of the earth's protective atmosphere are instances where acute radiation events can expose humans to radiation-based injuries. The long-term effects of acute radiation exposure include cataract formation, carcinogenesis, neurological degeneration, and other biomarkers of radiation-induced aging.

### SUMMARY OF THE INVENTION

Radiation-oxidative exposure treatment compositions comprise a mixture of micronutrient multivitamin and trace elements, a mixture of antioxidants and chemopreventative agents, and optionally a mixture of fatty acids.

Mixtures of micronutrient multivitamin and trace elements includes amounts of vitamin A, some of which is beta-carotine; vitamins Bp, B1, B2, B3, B5, B6, B7, B9, B12, C, D, E and K. The mixture also includes an amount of inositol. The mixture also includes amounts of calcium, iodine, magnesium, zinc, selenium, copper, manganese, chromium, molybdenum, potassium, boron and vanadium.

Mixtures of non-essential antioxidants and chemopreventative agents include bioflavins, which include rutin, quercetin, hesperidin; alpha lipoic acid (ALA), N-acetyl-L-cysteine (NAC), lutein, lycopene, astaxanthin, plant sterols, isoflavones, garlic extract, which provides allicin; green tea extract, which provides epigallocatech gallate; cruciferous vegetable extract, which provides glucosinolates; fruit extracts, coenzyme Q-10, and resveratrol.

Mixtures of fatty acids when included in a radiation-oxidative exposure treatment composition provides essential omega-3 fatty acids, including eicosapentaenoic acid (EPA) and docosahexaenoic acid (DHA).

In another embodiment there is disclosed a radiation-oxidative exposure treatment composition for ameliorating radiation-induced life shortening effects from exposure to a radiation source and the effects of oxidative stress such as premature skin aging due to sun exposure, exercise-induced muscle fatigue, oxidative stress of the liver due to toxic exposures, reduction in visual acuity, the radiation-oxidative exposure treatment composition comprising: (a) a mixture of micronutrient multivitamin and trace elements, (b) a mixture of antioxidants and chemopreventative agents, and (c) optionally, a mixture of fatty acids.

In one embodiment, the mixture of micronutrient multivitamin and trace elements may comprise: an amount of biotin in a range of from about 0.187 to about 0.45 mg; an amount of folate in a range of from about 0.25 to about 1.06 mg; an amount of iodine in a range of from about 0.0187 to about 2.07 mg; an amount of manganese in a range of from about 1.25 to about 7 mg; an amount of pantothenic acid in a range of from about 10 to about 25 mg; an amount of selenium in a range of from about 0.0875 to about 2.07 mg; an amount of vitamin B1 in a range of from about 1.5 to about 23.6 mg; an amount of vitamin B12 in a range of from about 0.0075 to about 0.009 mg; an amount of vitamin B2 in a range of from about 1.7 to about 10.4 mg; an amount of vitamin B3 in a range of from about 25 to about 50 mg; an amount of vitamin B6 in a range of from about 2 to about 43.6 mg; an amount of vitamin C in a range of from about 60 to about 500 mg; an amount of vitamin D in a range of from about 0.0125 to about 0.0445 mg; an amount of vitamin E in a range of from about 20 to about 643 mg; an amount of vitamin K in a range of from about 0.056 to about 0.080 mg; an amount of lycopene in a range of from about 1 to about 5 mg; an amount of zinc in a range of from about 15 to about 40 mg; an amount of alpha lipoic acid in a range of from about 100 to about 125 mg; an amount of astaxanthin in a range of from about 0.25 to about 0.31 mg; an amount of lutein in a range of from about 1 to about 6.25 mg; an amount of vitamin A in a range of from about 0.90 to about 1.85 mg; an amount of quercetin in a range of from about 0 to about 64 mg; an amount of glutathione in a range from about 0 to about 125 mg; an amount of pine bark extract in a range from about 0 to about 50 mg; an amount of curcumin extract in a range from about 0 to about 250 mg; an amount of calcium in a range of from about 0 to about 1,200 mg; an amount of chromium in a range of from about 0 to about 0.2 mg; an amount of copper in a range of from about 0 to about 5 mg; an amount of magnesium in a range of from about 0 to about 400 mg; an amount of molybdenum in a range of from about 0 to about 0.1 mg; an amount of inositol in a range of from about 0 to about 50 mg; and an amount of resveratrol in an amount of about 0 to about 5 mg.

In another embodiment, the mixture of micronutrient multivitamin and trace elements may comprise: biotin in an amount of about 0.187 mg; folate in an amount of about 0.25; iodine in an amount of about 0.0187 mg; manganese in an amount of about 1.25 mg; pantothenic acid in an amount of about 12.5 mg; selenium in an amount of about 0.0875 mg; vitamin B1 in an amount of about 1.9 mg; vitamin B12 in an amount of about 0.0075 mg; vitamin B2 in an amount of about 2.1 mg; vitamin B3 in an amount of about 25 mg; vitamin B6 in an amount of about 2.5 mg; vitamin C in an amount of about 75 mg; vitamin D in an amount of about 0.0125 mg; vitamin E in an amount of about 20 mg; vitamin K in an amount of about 0.056 mg; lycopene in an amount of about 1.25 mg; zinc in an amount of about 18.7 mg; alpha lipoic acid in an amount of about 125 mg; astaxanthin in an amount of about 0.31 mg; lutein in an amount of about 6.25 mg; vitamin A in an amount of about 0.938 mg; quercetin in an amount of about 64 mg; glutathione in an amount of about 125 mg; and where calcium, chromium, copper, magnesium, molybdenum, inositol and resveratrol are not present. In this embodiment, the glutathione may be replaced with either pine bark extract in an amount of about 20 mg, or curcumin extract in an amount of about 250 mg.

In yet another embodiment, the composition may further comprise: calcium in an amount of ≦about 250 mg; chromium in an amount of ≦about 0.2 mg; copper in an amount of ≦about 1 mg; magnesium in an amount of ≦about 100 mg; molybdenum in an amount of ≦about 0.056 mg; inositol in an amount of ≦about 50 mg; pine bark extract in an amount of ≦about 50 mg; and resveratrol in an amount of ≦about 5 mg; wherein the amount of quercetin is ≦about 64 mg; and wherein the amount of glutathione is ≦about 125 mg. In this embodiment, the glutathione may be replaced with either pine bark extract in an amount of ≦about 20 mg, or curcumin extract in an amount of ≦about 250 mg.

In still another embodiment, the mixture of micronutrient multivitamin and trace elements comprises: biotin in an amount of about 0.45 mg; folate in an amount of about 0.6; iodine in an amount of about 0.15 mg; manganese in an amount of about 4 mg; pantothenic acid in an amount of about 15 mg; selenium in an amount of about 0.1 mg; vitamin B1 in an amount of about 2.25 mg; vitamin B12 in an amount of about 0.009 mg; vitamin B2 in an amount of about 2.55 mg; vitamin B3 in an amount of about 30 mg; vitamin B6 in an amount of about 3 mg; vitamin C in an amount of about 150 mg; vitamin D in an amount of about 0.025 mg; vitamin E in an amount of about 66.7 mg; vitamin K in an amount of about 0.08 mg; lycopene in an amount of about 3 mg; zinc in an amount of about 15 mg; alpha lipoic acid in an amount of about 100 mg; astaxanthin in an amount of about 0.25 mg; lutein in an amount of about 5 mg; vitamin A in an amount of about 1.5 mg; quercetin in an amount of about 64 mg; glutathione in an amount of about 125 mg; and where calcium, chromium, copper, magnesium, molybdenum, inositol and resveratrol are not present. The glutathione may be replaced with either pine bark extract in an amount of about 20 mg, or curcumin extract in an amount of about 250 mg. In another embodiment, this composition may further comprise: calcium in an amount of about 250 mg; chromium in an amount of about 0.2 mg; copper in an amount of about 1 mg; magnesium in an amount of about 100 mg; molybdenum in an amount of about 0.056 mg; inositol in an amount of about 50 mg; pine bark extract in an amount of about 50 mg; and resveratrol in an amount of about 5 mg, and the glutathione could be replaced with either pine bark extract in an amount of about 20 mg, or curcumin extract in an amount of about 250 mg.

In yet another embodiment, the mixture of micronutrient multivitamin and trace elements comprises: vitamin A in an amount of about 5,000 IU (1.5 mg); vitamin C in an amount of about 150 mg; vitamin D in an amount of about 1,000 IU (0.025 mg); vitamin E in an amount of about 100 IU (100 mg); vitamin K in an amount of about 0.080 mg; vitamin B1 in an amount of about 2.25 mg; vitamin B2 in an amount of about 2.55 mg; vitamin B3 in an amount of about 30 mg; vitamin B6 in an amount of about 3 mg; folate in an amount of about 0.6 mg; vitamin B12 in an amount of about 0.009 mg; biotin in an amount of about 0.45 mg; calcium in an amount of about 250 mg; iodine in an amount of about 0.15 mg; magnesium in an amount of about 100 mg; zinc in an amount of about 15 mg; selenium in an amount of about 0.1 mg; copper in an amount of about 1 mg; manganese in an amount of about 4 mg; chromium in an amount of about 0.2 mg; molybdenum in an amount of about 0.056 mg; boron in an amount of about 1 mg; and vanadium in an amount of about 0.05 mg. In this embodiment, the composition could further comprise: alpha lipoic acid in an amount of about 100 mg; citrus bioflavonoids in an amount of about 64 mg; pine bark extract in an amount of about 50 mg; resveratrol in an amount of about 5 mg; lutein in an amount of about 5 mg; lycopene in an amount of about 3 mg; and astaxanthin in an amount of about 0.25 mg, where the composition could further comprise: Resveratrol in an amount of about 5 mg; N-acetyl cysteine in any amount; and Beta-carotene in any amount up to the total claim for vitamin A. The N-acetyl cysteine could be replaced with glutathione or curcumin extract in any amount that is generally recognized as safe.

Methods of treatment of a subject exposed to a radiation source or an oxidative stress, or both, with the radiation-oxidative exposure treatment composition include the step of administering to the subject a daily dose of the radiation-oxidative exposure treatment composition such that the life shortening effects induced by the radiation source or the oxidative stress are ameliorated.

In some methods the administration of the daily dose of the radiation-oxidative exposure treatment composition occurs on a continuing daily basis for at least 7 days before exposure to the radiation source or oxidative stress. In some other methods, the administration of the daily dose of the radiation-oxidative exposure treatment composition occurs on a continuing daily basis after exposure to the radiation source or oxidative stress.

Some methods include the step of administering to the subject an amount of manganese superoxide dismutase (MnSOD) plasmid DNA in liposome at least 24 hours before exposure to the radiation source.

The daily dose of radiation-oxidative exposure treatment composition can be administered proportionally during the 24-hour period such that the sum of the proportional amounts totals the daily dose.

### BRIEF DESCRIPTION OF THE DRAWINGS

These and other features, aspects, and advantages of the present invention are better understood with regard to the following Detailed Description of the Preferred Embodiments, appended Claims, and accompanying Figures, where:

FIG. 1 is a graph showing percentage overall survival of the members of four groups of mice receiving 9.5 Gy of radiation for a period of 450 days after initial exposure; and

FIG. 2 is a graph showing percentage condition survival of the of the members of the four groups of mice receiving 9.5 Gy of radiation for the period of 30 days after initial exposure to 450 days after initial exposure.

### DETAILED DESCRIPTION OF THE PREFERRED EMBODIMENTS

The Specification, which includes the Summary of Invention, Brief Description of the Drawings and the Detailed Description of the Preferred Embodiments, and the appended Claims refer to particular features (including method steps) of the invention. Those of skill in the art understand that the invention includes all possible combinations and uses of particular features described in the Specification, including all of those features specifically described. For example, in describing a feature as part of an embodiment or an aspect of the invention, one of ordinary skill in the art understands that the described feature can and is used, to the extent possible, in combination with or in context of other features described as part of other embodiments and aspects of the invention.

Those of skill in the art understand that the invention is not limited to or by the description of embodiments as given in the Specification. Those of skill in the art also understand that the terminology used for describing particular embodiments does not limit the scope or breadth of the invention.

### Problem

Because acute radiation sickness (ARS) occurs within a very short period, the opportunity to treat or mitigate the effects of high-dose irradiation is limited. As an augmentation to treatment, prophylactic measures can be a more effective strategy to address acute radiation-induced phenomenon. Preventing the onset of ARS may also minimize other biological consequences of ionizing radiation, which is an additional benefit.

Developing countermeasures for radiation injury has a long history and is very challenging. Joint research with NASA has postulated that that the era of high-dose single counter-radiation agents is ending. Development of a multi-pathway defense strategy via comprehensive dietary ingredient cocktail is a successful approach to protect the human body against either acute or chronic sources of oxidative damage or radiation exposure. Oxidative damage in humans working or living in extreme environments is widespread and affects many cellular components. Clinical research shows downstream biological effects from this damage are variable, based upon host factors, dose quality, magnitude and rate, as well as the presence or absence of countermeasures.

There is accumulating evidence for a role of oxidative stress in both the acute and chronic effects of ionizing radiation. Administration of organ-specific targeted antioxidant therapies, including manganese superoxide dismutase plasmid DNA in liposome (MnSOD-PL) gene product, can increase survival rates due to a decrease in acute and chronic toxicities of single-fraction and fractionated irradiation. Systemic administration of antioxidant agents, including amifostine, GS-nitroxide and superoxide dismutases (SODs), also decreases acute and chronic toxicities.

With respect to late effects of ionizing radiation, two categories of studies exist. Prior studies report improved conditional survival of MnSOD-PL-treated high-dose-irradiated animals for acute radiation events. Other studies describe improved conditional survival effects of antioxidants in low-doses or partial-body-irradiated animals; however, these studies use very high dosages of antioxidants such that they are toxic to the subject.

### Solution

Certain antioxidants (e.g., α-tocopherol, ascorbic acid, beta-carotene, etc.), have properties that protect cells from oxygen free-radical toxicity, and therefore can decrease the type of oxidative damage observed among subjects exposed to radiation, particularly astronauts exposed to radiation or hypobaric hyperoxia. Additionally, antioxidants can reduce oxidative damage associated with prolonged hyperoxic environments, among other culprits of oxidative damage.

Vitamin C is a potent antioxidant capable of reversing endothelial dysfunction caused by increased oxidant stress. Though it seems likely that vitamin C supplementation would mitigate hyperoxia-induced oxidative damage among extravehicular activities (EVA), it is debated whether vitamin C could act as a pro-oxidant when iron stores are elevated. Vitamin C can also act as a pro-oxidant in large doses as a single-agent. Treatments with vitamin A, C, or E can protect rats exposed to acute hyperoxia (80% oxygen) against oxygen toxicity by elevating glutathione concentration. Vitamin E supplementation to rabbits can decrease lipid peroxidation and diminish increases in pulmonary antioxidant enzymes induced by in vitro 100% oxygen exposure. These increases likely contribute to symptoms of oxidative stress. α-tocopherol is also effective in preventing hyperoxia-induced DNA fragmentation and apoptosis. Flavonoids appear to exhibit more antioxidant effects than α-tocopherol in healthy adults. In addition to a plethora of other tested agents (e.g., a-lipoic acid, folic acid, co-enzyme Q10, selenium, beta carotene, glutathione, and N-acetylcysteine), there are a large number of plant extracts that have antioxidant properties, including strawberry and blueberry, curcumin, and green tea. The tested agents can be provided in an isolated form, or provided via another substance that contains the agent, for example, yeast as a glutathione-containing substance.

Quercetin, a plant bioflavonoid, appears to be a powerful antioxidant and free radical scavenger while also demonstrating desirable anti-carcinogenic, neuroprotective, anti-viral, and cardio/vascular protective properties. Quercetin also appears to help prevent cataract formation and exhibit positive effects on cognitive performance and immune response. In vitro experiments suggest it may be beneficial in protecting against bone loss. Furthermore, recent studies suggest having a protective mechanism against viral illness after exertional stress in athletes and synergistic properties with other micronutrients such as Vitamin C, B3, and omega-3 fatty acids.

Additionally, supplementing animals exposed to carcinogens and ionizing radiation with omega-3 fatty acids and fiber can reduce the risk of cancer. Omega-3 fatty acids show a benefit of improving lipid parameters in those individuals with unfavorable total cholesterol to high-density lipoprotein ratios. Combinations of docosahexaenoic acid (DHA), eicosapentaenoic acid (EPA) and other fatty acids appear to show efficacy in improving cognitive performance and mood in test subjects with affective disorders, traumatic brain injury, and exposure to environmental stress.

Oxidative stress may be involved in the pathogenesis of several conditions leading to declining functionality, both in normal as well as diseased individuals. Dietary antioxidants can play a role in neutralizing free radicals caused by factors including exposure to radiation.

### Radiation-Oxidative Exposure Treatment Compositions

Compositions comprising low levels of each of the most effective micronutrient multivitamins, trace elements, antioxidants, chemoprevention agents and optionally certain fatty acids, allows for a broad range of cellular protection and bioavailability without the toxicity usually associated with high single doses of particular vitamins, elements, antioxidants, chemoprevention agents, and lipids.

Radiation-oxidative exposure treatment compositions comprise a mixture of micronutrient multivitamin and trace elements, a mixture of antioxidants and chemopreventative agents, and optionally a mixture of fatty acids.

The radiation-oxidative exposure treatment compositions include a mixture of micronutrient multivitamins and trace elements. The low levels of each of the most effective protection molecules allows delivery to a subject, such as a human, without the toxicity associated with high-dose, oral single agents, and with conceivably better efficacy. Most micronutrient multivitamins and trace elements are at the levels of federally Recommended Daily Allowance. Some vitamins with antioxidant capacity are at slightly higher but safe dosage levels (i.e., well below levels of any adverse effect).

The radiation-oxidative exposure treatment compositions comprise a mixture of antioxidants and chemopreventative agents. The non-essential natural antioxidants and chemoprevention agents derive from natural foods and herbal sources. Many of the nonessential natural antioxidants and chemoprevention agents demonstrate antioxidant effects. Previous studies in scientific peer-reviewed journals report doses as such safe, including the NIH consensus conference on dietary supplements. Recommendations by the National Cancer Institute/Chemoprevention Branch for possible reductions in cancer development risk, epidemiological reviews, and testing in randomized, placebo-controlled studies provide additional support for their safe use.

Optionally, the radiation-oxidative exposure treatment compositions include a mixture comprising fatty acids, including omega-3 fatty acids. Fatty acids, specifically fatty acids obtained from fish oil, have been found to have a number of beneficial health effects. It is understood that oil from fish contains EPA and DHA. These are classified as omega-3 fatty acids. These omega-3 fatty acids derived from fish oil are known to keep blood triglycerides in check and may inhibit the progression of atherosclerosis. EPA and DHA are believed to have anti-inflammatory activity and are sometimes used as dietary supplements with inflammatory conditions, such as Crohn's disease and rheumatoid arthritis. It is believed that the omega-3 fish oil fatty acids may balance other fatty acids. When fatty acids are out of balance in the body, the body may release chemicals that promote inflammation. Omega-3 fatty acids are needed for prostaglandin. Prostaglandins are hormone-like substances that regulate dilation of blood vessels, inflammatory responses, and other critical body processes. DHA and EPA are also believed essential for nerve and eye functions. DHA comprises about 60 percent of the outer rod segments of photoreceptor cells that are used to see with by humans. Brain tissue has a substantial component of fat composed of DHA. It is believed that fish oil omega-3 fatty acids and, specifically, DHA and EPA, are useful in wet macular degeneration since these fatty acids help heal and support blood vessel walls. Studies show that eating fish several times a month may reduce the risk of developing AMD.

Pharmacopeial compendia, including the United States Pharmacopeia and National Formulary (USP 32-NF 27), give the materials and specifications for micronutrient vitamins (e.g., ascorbic acid, cholecalciferol), trace elements (e.g., potassium, zinc), and other coenzyme and non-botanical constituents (e.g., coenzyme Q-10, choline bitartrate, N-acetyl cysteine) for the radiation exposure treatment compositions.

The supplier's specifications and current Good Manufacturing Practices (cGMP) provide the standardized protocols for extracting, isolating, or producing ingredients of a botanical nature not subject to pharmacopeial monographs (e.g., quercetin, astaxanthin, fruit extracts).

All starting, intermediate and finished materials are appropriate for food use. U.S. Food and Drug Administration lists all the starting, intermediate, and final materials as “GRAS” (Generally Recognized as Safe).

The supplier verifies each mixture comprising micronutrient multivitamin and trace elements, antioxidants and chemopreventative agents, and fatty acids for homogeneity, assay, particle size, microbial specifications, density, humidity and other applicable measures of quality.

### Micronutrient Vitamin and Trace Element Mixtures

The first mixture comprises micronutrient vitamins and trace elements. The first dietary supplement can contain various vitamins important for the dietary requirement of animals, including mammals, and especially humans (homo sapiens), including Vitamins A, Bp, B1, B2, B3, B5, B6, B7, B9, B12, C, D, E and K. Some of the vitamins also have antioxidant properties.

There may be more than one source for micronutrient vitamins. Vitamin A palmitate and beta-carotene, and combinations of the two, are sources of Vitamin A. Choline bitartrate is a source of choline. Ascorbic acid is a source of Vitamin C. Sodium ascorbate is also a source for Vitamin C. Cholecalciferol is a source of Vitamin D. D-alpha tocopheryl succinate and mixed tocopherols, and combinations of the two, are sources of Vitamin E. Natural and mixed carotenoids are preferred sources of Vitamin E. Phytonadione is a source of Vitamin K. Thiamine can originate from thiamine mononitrate, which provides Vitamin B 1. Riboflavin is a source of Vitamin B2. Niacin can originate from inositol hexanicotinate, which provides Vitamin B3. Pyridoxine hydrochloride is a source of Vitamin B6. Folate can originate from folic acid, which provides Vitamin B9. Cyanocobalamin is a source of Vitamin B12. Biotin is a source of B7. Pantothenic acid can originate from d-calcium pantothenate, which provides Vitamin B5.

The first dietary supplement also contains inositol. Although no longer considered a Vitamin B complex on its own, many vitamin supplement formulations still include inositol for its general bioactivity. Inositol hexanicotinate is the niacin-esterified version of inositol. Inositol and inositol hexanicotinate, and combinations of the two, can provide inositol.

The first dietary supplement can also contain various trace elements important for the dietary requirement of mammals, especially humans, including calcium, iodine, magnesium, zinc, selenium, copper, manganese, chromium, molybdenum, potassium, boron and vanadium.

There may be more than one source for trace elements. Calcium carbonate and dicalcium phosphate, and combinations of the two, are sources of calcium. Kelp is a source of iodine. Magnesium oxide and chelate, and combinations of the two, are sources of magnesium. Zinc chelate [monomethionine], zinc oxide and zinc gluconate are sources of zinc. Zinc oxide provides the most concentrated form of elemental zinc. 1-Selenomethionine is a source of selenium. Copper amino acid chelate, copper oxide and copper gluconate are sources of copper. Manganese amino acid chelate is a source of manganese. Chromium polynicotinate is a source of chromium. Molybdenum amino acid chelate is a source of molybdenum. Potassium citrate is a source of potassium. Boron chelate is a source of boron. Vanadyl sulfate is a source of vanadium.

Units of measure for Tables 1-6 include “IU”, which represents “International Units”, an understood metric in the art for measuring the active amount of particular species, especially vitamins (e.g., Vitamins A, D, and E). Milligrams (“mg”) are 1×10-3 grams. Micrograms (“μg”) are 1×10-6 grams.

Table 1 shows the composition range of components for useful micronutrient multivitamin and trace element mixtures for use with the daily dose radiation and oxidative exposure treatment compositions. Table 2 shows the daily dose of a useful mixture of micronutrient multivitamins and trace elements for use with radiation and oxidative exposure treatment compositions.

TABLE 1
Composition range for daily doses of useful
micronutrient multivitamin and trace
element mixtures for use with radiation and oxidative
exposure treatment compositions.

| | Daily Dose | Units of |
|---|---|---|
| Ingredient | Range | Measure |
| Total Vitamin A | 2500-10000 | IU |
| Vitamin A (pre-formed) | 0-1000 | IU |
| Beta-carotene (as part of total Vitamin A) | 0-1000 | IU |
| Vitamin C | 60-500 | mg |
| Vitamin D | 400-2000 | IU |
| Vitamin E | 30-400 | IU |
| Vitamin K | 45-85 | Mg |
| Thiamine (Vitamin B1) | 1.5-50 | Mg |
| Riboflavin (Vitamin B2) | 1.7-50 | mg |
| Niacin (as inositol hexanicotinate, niacin or | 20-50 | mg |
| niacinamide) | | |
| Vitamin B6 | 2-50 | mg |
| Folate | 200-800 | μg |
| Vitamin B12 | 6-50 | μg |
| Biotin | 150-1000 | μg |
| Pantothenic acid | 10-100 | mg |
| Calcium | 0-1200 | mg |
| Iodine | 15-130000 | μg |
| Magnesium | 0-400 | mg |
| Zinc | 15-80 | mg |
| Selenium | 70-200 | μg |
| Copper | 0-5 | mg |
| Manganese | 1-10 | mg |
| Chromium | 0-600 | μg |
| Molybdenum | 0-100 | μg |
| Potassium (as potassium citrate) | 0-3500 | mg |
| Choline (as choline bitartrate) | 0-500 | mg |
| Inositol | 0-300 | mg |
| Boron | 0-5 | mg |
| Vanadium | 0-300 | μg |

TABLE 2
Daily dose of a useful mixture of micronutrient
multivitamins and trace elements for
use with radiation and oxidative exposure
treatment compositions.

| | Daily | Units of |
|---|---|---|
| Ingredient | Dose | Measure |
| Vitamin A (70% beta-carotene and | 250000 | IU |
| 30% vitamin A palmitate) | | |
| Vitamin C (as ascorbic acid | 250 | mg |
| Vitamin D (as cholecalciferol) | 1200 | IU |
| Vitamin E (as natural d-alpha tocopherol succinate and | 200 | IU |
| mixed tocopherols) | | |
| Vitamin K (as phytonadione) | 80 | μg |
| Thiamine (vitamin B1) (as thiamine mononitrate) | 1.5-50 | mg |
| Riboflavin (Vitamin B2) | 2.25 | mg |
| Niacin (as inositol hexanicotinate) | 30 | mg |
| Vitamin B6 (as pyridoxine hydrochloride) | 3 | mg |
| Folate (as folic acid) | 600 | μg |
| Vitamin B12 (as cyanocobalamin) | 9 | μg |
| Biotin | 450 | μg |
| Pantothenic acid (as d-calcium pantothenate) | 15 | mg |
| Calcium (as calcium carbonate, dicalcium phosphate) | 500 | mg |
| Iodine (from kelp) | 30 | μg |
| Magnesium (as magnesium oxide and chelate) | 200 | mg |
| Zinc (as zinc chelate [monomethionine or glycinate]) | 15 | mg |
| Selenium (as L-selenomethionine) | 100 | μg |
| Copper (as copper amino acid chelate) | 0.18 | mg |
| Manganese (as manganese amino acid chelate) | 2 | mg |
| Chromium (as chromium picolinate) | 200 | μg |
| Molybdenum (as molybdenum amino acid chelate) | 56 | μg |
| Potassium (as potassium citrate) (7.5 mEg) | 290 | mg |
| Choline | 50 | mg |
| Inositol | 50 | mg |
| Boron (as boron chelate) | 1 | mg |
| Vanadium (as vanadyl sulfate) | 50 | μg |

In some embodiment mixtures of micronutrient multivitamins and trace elements, the amount of Vitamin A for the daily dose is about 750 IU.

Vitamin C is arguably the most important water-soluble biological antioxidant. It can scavenge both reactive oxygen species (ROS) and reactive nitrogen species thought to play roles in tissue injury associated with the pathogenesis of various conditions. By virtue of this activity, it inhibits lipid peroxidation, oxidative DNA damage and oxidative protein damage. It helps preserve intracellular reduced glutathione concentrations, which in turn helps maintain nitric oxide levels and potentiates its vasoactive effects. In addition, vitamin C may modulate prostaglandin synthesis to favor the production of eicosanoids with antithrombotic and vasodilatory activity.

The mechanisms underlying the immune effects of zinc are not fully understood, though some of them may be accounted for by its membrane-stabilization effect. Zinc is also believed to have secondary antioxidant activity. Although zinc does not have any direct redox activity under physiological conditions, it nevertheless may influence membrane structure by its ability to stabilize thiol groups and phospholipids. It may also occupy sites that might otherwise contain redox active metals such as iron. These effects may protect membranes against oxidative damage. Zinc also comprises the structure of copper/zinc superoxide dismutase (Cu/Zn SOD), a very powerful antioxidant. Additionally, it may have secondary antioxidant activity via the copper-binding protein metallothionein.

Vitamin A (retinyl palmitate ester) is hydrolyzed by a pancreatic hydrolase and combined with bile acids and other fats prior to its uptake by enterocytes in the form of micelles. It is then re-esterified and secreted by the enterocytes into the lymphatic system in the form of chylomicrons. These chylomicrons enter the circulation via the thoracic duct and undergo metabolism via lipoprotein lipase. Most of the retinyl esters are then rapidly taken up into liver parenchymal cells and again hydrolyzed to all-trans retinol and fatty acids (e.g., palmitate). All-trans retinol may be then stored by the liver as retinyl esters or transported in the circulation bound to serum retinol binding protein (RBP). Serum RBP is the principal carrier of retinol, which comprises greater than 90% of serum vitamin A. It is believed that RBP in association with transthyretin or prealbumin co-transport proteins are responsible for the transport of retinol into target cells. All-trans retinol is delivered to the cornea via the tears and by diffusion through eye tissue. Retinol is oxidized to retinal via retinol dehydrogenase. Retinal is metabolized to retinoic acid via retinal dehydrogenase. The metabolites of retinol and retinoic acid undergo gucuronidation, glucosylation and amino acylation. They are excreted mainly via the biliary route, though some excretion of retinol and its metabolites also occurs via the kidneys.

Intestinal absorption of vitamin C occurs primarily via a sodium-dependent active transport process, although some diffusion may also come into play. The major intestinal transporter is SVCT1 (sodium-dependent vitamin C transporter 1). Some ascorbic acid may be oxidized to dehydroascorbic (DHAA) acid and transported into enterocytes via glucose transporters. Within the enterocytes, all DHAA is reduced to ascorbic acid via reduced glutathione, and ascorbic acid leaves the enterocytes to enter the portal and systemic circulation for distribution throughout the body. The transporter SVCT2 appears to aid in the transport of vitamin C into the aqueous humor of the eyes. Though it cannot itself cross the blood-brain barrier, ascorbic acid may be oxidized to DHAA and be transported to the brain tissues via GLUT1 (glucose transporter 1), where it can then be reduced back to ascorbic acid for utilization. Metabolism and excretion of vitamin C occurs primarily via oxidation to DHAA and hydrolyzation to diketogulonate, though other metabolites such as oxalic acid, threonic acid, L-xylose and ascorbate-2-sulfate can also result. The principal route of excretion is via the kidneys.

Vitamin D is principally absorbed in the small intestine via passive diffusion. It is delivered to the enterocytes in micelles formed from bile acids, fats, and other substances. Like vitamin A, vitamin D is secreted by the enterocytes into the lymphatic system in the form of chylomicrons and enters the circulation via the thoracic duct. It is also transported in the blood bound to an alpha globulin known as Vitamin D-Binding Protein (DBP) and the group-specific component (Gc) protein. Much of the circulating vitamin D is extracted by the hepatocytes to be metabolized to 25-hydroxyvitamin D [25(OH)D] or calcidiol via the enzyme vitamin D 25-hydroxylase. 25(OH)D is then metabolized in the kidney to the biologically active hormone form of vitamin D, calcitrol [1,25(OH)2D], via the enzyme 25-hydroxyvitamin D-1-alphahydroxylase. Calcitrol may undergo further hydroxylation and metabolism into 24,25(OH)2D and 1,24,25(OH)3D. These metabolites, as well as vitamin D are excreted primarily via the biliary route. The final degradation product of 1,25(OH)2D is calcitroic acid, which is excreted by the kidney.

Much of the pharmacokinetics of zinc in humans remains unknown. Zinc is absorbed all along the small intestine, though most appears to be assimilated from the jejunum. Zinc uptake across the brush border appears to occur by both a saturable barrier-mediated mechanism and a non-saturable non-mediated mechanism. The exact mechanism of zinc amino-acid chelates (such as the zinc-methionine used in AmeriSciences OS2) transport into the enterocytes remains unclear, but evidence demonstrates greater bioavailability than other supplemental forms. Zinc transporters have been identified in animal models. Once the mineral is within the enterocytes, it can be used for zinc-dependent processes, become bound to metallothionein and held within the enterocytes or pass through the cell. Transport of zinc across the serosal membrane is carrier-mediated and energy-dependent. Zinc is transported to the liver via the portal circulation. A fraction of zinc is extracted by the hepatocytes, and the remaining zinc is transported to the various cells of the body via the systemic circulation. It is transported bound to albumin (about 80%), alpha-3-macroglobulin (about 18%), and to such proteins as transferin and ceruloplasmin The major route of zinc excretion appears to be the gastrointestinal tract via biliary, pancreatic or other gastrointestinal secretions. Fecal zinc is also comprised of unabsorbed dietary zinc as well as the sloughing of mucosal cells.

### Antioxidant and Chemopreventative Agent Mixtures

The antioxidant and chemopreventative agent mixture is a combination of botanical extracts, carotenoids, flavonoids, and other ancillary compounds, which can provide antioxidant activity and some measure of protection against oxidative stress.

Antioxidant and chemopreventative agent mixtures contain non-essential natural antioxidants and chemopreventative agents, including rutin, quercetin, hesperidin, alpha lipoic acid (ALA), N-acetyl-L-cysteine (NAC), lutein, lycopene, astaxanthin, plant sterols, isoflavones, garlic extract, green tea extract, cruciferous vegetables (and their extracts), fruit blends, coenzyme Q-10, and resveratrol. It may also contain other similar non-essential antioxidant ingredients that are botanical, nutritional, dietary, additive, or otherwise Generally Recognized as Safe (GRAS) and allowed within the applicable jurisdiction, and not already listed herein, such as Glutathione, Curcumin, Acai, Caffeine, etc. Soy extract is a source for isoflavones. Bulb garlic is a source for garlic extract. Green tea leaf is a source for green tea extract and epigallocatech gallate. The green tea leaf extract is standardized to 95% polyphenols and 50% epigallocatech gallate (EGCG). Broccoli sprouts are a source for cruciferous vegetable extract. Strawberries, escobillo, blueberries, blackberries, cranberries, grapes, and pomegranates are sources for fruit blends.

Quercetin, rutin and hesperidin are flavonols with a phenyl benzo(c)pyrone-derived structure. Extraction of the quercetin glycosides, primarily rutin, from plants, produces commercial quantities of quercetin. Citrus peel, apples, onions and Uncaria leaves are useful for the isolation and synthesis of quercetin. Preferably, the starting material for the flavonols for the non-essential natural antioxidants and chemoprevention agents is immature sun-dried Fava d'Anta beans (Dimorphandra mollis or Dimorphandra gardeneriana). The manufacturing process for quercetin includes the aqueous extraction of rutin from the plant source, release of the aglycone via hydrolysis through the addition of an acidic aqueous solution, and neutralization to produce a crude crystalline quercetin product. Several purification processes to the resultant quercetin product yields purified quercetin crystals.

Green tea extract originates from the leaves of Camellia sinensis. Gently washing, drying, shivering, compacting and keeping the leaves at controlled room temperature under low humidity conditions occurs prior to extract processing. Extraction takes place in a reactor using purified water at about 90° C. In one embodiment, processing at high pressure and lower temperatures can concentrate the intermediate extraction product. In another embodiment, food processing appropriate solvents may be employed to assist in providing a filtered and crystallized extract. In another embodiment, drying and powdering to specification may be used to complete the production process.

Antioxidant and chemoprevention agent mixtures contain a blend of fruit concentrates and extracts having elevated antioxidant values. The U.S. Department of Agriculture's Database for the Oxygen Radical Absorbance Capacity (ORAC) lists antioxidant values. Processing whole fruits of F. ananassa (strawberry), E. vaccinium (blueberry), R. rubus (blackberry) and E. vaccinium (cranberry) for use in the non-essential natural antioxidants and chemoprevention agents mixture includes washing and treating only with water. Drying and blending into powdered fruit concentrates completes the processing of the fruits.

Percolation processes can produce extracts from M. glabra (Escobillo), V. vinifera (grape) and P. granatum (pomegranate) using solutions of water, ethanol or combinations of both as a solvent. In one embodiment, homogenization of the extracts may be achieved in a two-stage process with heated transfer lines. A spray dry tower may be used to powder the extracts.

All fruit-sourced materials undergo visual inspection and metal detection scanning before blending and combination.

Brassica oleracea italia seed has perceived health benefits and high antioxidant values attributed to its content of sulforaphane. Collections of the seeds are the precursor for growing and cultivating broccoli sprouts in pesticide-free conditions. The harvesting of florets of young broccoli occurs to maximize glucosinolate content. Processing technology controls endogenous myrosinase enzymes to prevent sulforaphane digestion. The process does not use solvents. Approximately 20 pounds of broccoli sprouts yield 1 pound of cruciferous vegetable extract material (i.e., a 20:1 concentration).

Resveratrol (3,4′,5-trihydroxystilbene) is a polyphenolic compound of the class of stilbenes. Some types of plants produce resveratrol and other stilbenes in response to stress, injury, fungal infection and ultraviolet (UV) irradiation. Resveratrol-3-Obeta-glucoside is a piceid. Vitis vinifera, Carignane and Cinsault varieties are whole red grapes from the Rhone Valley in Southern France. Grape seeds and skins collected from wine fermentation vessels form the extraction material. A multistep process involving water extraction and purification of polyphenols on adsorbent resin ensures high purity and reproducibility. Prior to blending and release, standardization, quality assurance testing and metal detection scanning occurs. Approximately 500 to 750 pounds of red grapes yields 1 pound of the standardized extract.

Isoflavones are polyphenolic compounds commonly found in legumes, including soybeans. The most common and abundant soy isoflavone aglycone is genistein, followed by daidzein and glycitein. The soy isoflavone isolate starts off with non-GMO soybeans that undergo extraction with water and ethanol, filtration, elution with a resin, concentration and a second round of filtration. Drying, pulverizing, assaying, diluting, and blending the extract achieves standardization specifications.

Astaxanthin is a carotenoid with known antioxidant properties and documented effects on immunology, muscular endurance, visual acuity, reduced rate of macular degeneration, and reactive oxygen species (ROS). The algae Haematococcus pluvialis, cultivated in Hawai'i, is a starting material for astaxanthin extract. Washing, drying, and pulverizing occur after harvesting. Effused supercritical CO2 extracts a dried biomass intermediate. The product forms from mixing the resulting oleoresin extract intermediate with stabilizing ingredients generally recognized by the Food and Drug Administration and then spray dried. Milling and chilsonating the end product occurs to the specified mesh size to finish the product.

Table 3 shows the composition range of components for useful antioxidant and chemopreventative agent mixtures for use with radiation and oxidative exposure treatment compositions. Table 4 shows the daily dose of a useful mixture of antioxidant and chemopreventative agent mixtures for use with radiation and oxidative exposure treatment compositions.

TABLE 3
Composition range for daily doses of useful antioxidant
and chemopreventative agent
mixtures for use with radiation and oxidative
exposure treatment compositions

| | Daily Dose | Units of |
|---|---|---|
| Ingredient | Range | Measure |
| Total bioflavonoids (including, for | 50-1000 | mg |
| example, quercetin, | | |
| rutin, hesperidin) | | |
| Rutin | 0-500 | mg |
| Quercetin | 50-1000 | mg |
| Hesperidin | 0-500 | mg |
| Alpha lipoic acid | 100-1000 | mg |
| N-acetyl-L-cysteine (NAC) | 0-1000 | mg |
| (optional) | | |
| Lutein | 5-15 | mg |
| Lycopene | 1-10 | mg |
| Astaxanthin | 0.25-10 | mg |
| Plant sterols and/or sterols (free or | 0-1000 | mg |
| esterified) | | |
| Soy isoflavones | 0-350 | mg |
| Garlic extract (bulb) | 0-500 | mg |
| Allicin (garlic extract) | 0-13 | mg |
| Green tea extract (leaf) | 0-1000 | mg |
| Epigallocatechin Gallate (EGCG) (from | ≦5000 | mg |
| green tea extract) | | |
| Cruciferous vegetables or vegetable extract | ≦5000 | mg |
| (Brassica spp.) | | |
| Mixed fruit extract (strawberry, escobillo, | ≦5000 | mg |
| blueberry, blackberry, cranberry, grape, | | |
| and/or pomegranate) | | |
| Coenzyme Q-10 | 0-240 | mg |
| Resveratrol | ≦150 | mg |

TABLE 4
Daily dose of a useful mixture of antioxidant
and chemopreventative agent mixtures for
use with radiation and oxidative exposure treatment compositions.

| | Daily | Units of |
|---|---|---|
| Ingredient | Dose | Measure |
| Quercetin (as quercetin dihydrate and/or citrus peel) | 800 | mg |
| Rutin (citrus peel) | 25 | mg |
| Hesperidin (citrus peel) | 5 | mg |
| Green Tea Polyphenols (green tea extract (leaf)) | 450 | mg |
| Epigallocatechin Gallate (EGCG) (green tea extract) | 250 | mg |
| Alpha lipoic acid | 400 | mg |
| N-acetyl-L-cysteine (NAC) | 600 | mg |
| (optional) | | |
| Lycopene (tomato extract 5%) | 5 | mg |
| Astaxanthin (Haematococcus Algae Extract 2%) | 1 | mg |
| Lutein (Marygold Extract 5%) | 10 | mg |
| Phytosterols (Soy and Avocado) | 250 | mg |
| Isoflavones (Soy and/or Avocado Extracts) | 25 | mg |
| High-Potency Garlic Extract (bulb) | 275 | mg |
| Allicin (from garlic extract) | 7.25 | mg |
| Cruciferous Vegetable (Brassica spp.) (plant)) | 100 | mg |
| Glucosinolates (from cruciferous veg. extract) | 4 | mg |
| High ORAC fruit extract (strawberry, escobillo, | 100 | mg |
| blueberry, blackberry, cranberry, grape, pomegranate) | | |
| Coenzyme Q-10 | 100 | mg |
| Resveratrol (phytoalexin from grape juice/seed extract, | 5 | mg |
| polygonum extract, or other source) | | |

In some embodiment mixtures of antioxidants and chemopreventative agents, the amount of green tea extract for the daily dose is about 250 mg.

Astaxanthin has both lipo- and hydrophilic antioxidant activity, working both inside as well as outside cell membranes. Astaxanthin is known to cross the blood-brain barrier and effectively work inside retinal tissues. Evidence suggests it inhibits the neurotoxicity induced by peroxide radicals or serum deprivation; reduces the intracellular oxidation induced by various reactive oxygen species (ROS). Furthermore, astaxanthin reduced the expressions of 4-hydroxy-2-nonenal (4-HNE)-modified protein (indicator of lipid peroxidation) and 8-hydroxydeoxyguanosine (8-OHdG; indicator of oxidative DNA damage) in animal models. These findings indicate that astaxanthin has positive effects against cellular damage in-vivo, and that its protective effects may be partly mediated via its antioxidant effects.

Alpha-lipoic acid (ALA) forms a redox couple with its metabolite, dihydrolipoic acid (DHLA) and may scavenge a wide range of reactive oxygen species. Both ALA and DHLA can scavenge hydroxyl radicals, nitric oxide radicals, peroxynitrite, hydrogen peroxide and hypochlorite. ALA, but not DHLA, may scavenge singlet oxygen, and DHLA, but not ALA, may scavenge superoxide and peroxyl reactive oxygen species.

ALA has been found to decrease urinary isoprostanes, O-LDL and plasma protein carbonyls, markers of oxidative stress. Furthermore, ALA and DHLA have been found to have antioxidant activity in aqueous as well as lipophilic regions, and in both extracellular as well as intracellular environments. ALA is also involved in the recycling of other biological antioxidants such as vitamins C and E, as well as glutathione.

Alpha lipoic acid pharmacokinetic data demonstrate that its absorption takes place from the small intestine, followed by portal circulation delivery to the liver, and to various tissues in the body via systemic circulation. Alpha lipoic acid readily crosses the blood brain barrier, and is readily found (following distribution to the various tissues) extracellularly, intracellularly and intramitochondrially. It is metabolized to its reduced form, dihydrolipoic acid (DHLA) by mitochondrial lipoamide dehydrogenase, which can in turn form a redox couple with lipoic acid. ALA is also metabolized to lipoamide, which forms an important cofactor in the multienzyme complexes that catalyze pyruvate and alpha-ketoglutarate, both important aspects of cellular respiration and energy production via the Krebs cycle. ALA can also be metabolized to dithiol octanoic acid, which can undergo catabolism.

Carotenoids such as lutein and zeaxanthin appear to be more efficiently absorbed when administered with high-fat meals. They are hydrolyzed in the small intestine via esterates and lipases, and solubilized in the lipid core of micelles formed from bile acids and other lipids. They can also form clathrate complexes with conjugated bile salts. Both of these complexes can deliver carotenoids to the enterocytes, where they are then released into the lymphatics in the form of chylomicrons. From there, they are transported to the general circulation via the thoracic duct. Lipoprotein lipases hydrolyze much of the triglyceride content in the chylomicrons found in the circulation, resulting in the formation of chylomicrons remnants, which in turn retain apolipoproteins E and B48 on their surfaces and are mainly taken up by the hepatocytes. Within the liver, carotenoids are incorporated into lipoproteins and they appear to be released into the blood mainly in the form of HDL and—to a much lesser extent—VLDL. Astaxanthin is distributed throughout the body, with muscle tissue seemingly receiving larger concentrations based on tissue/plasma ratio at 8 and 24 hours after oral ingestion. Lutein appears to undergo some metabolism in-situ to meso-zeaxathin. Xanthophylls as well as their metabolites are believed to be excreted via the bile and, to a lesser extent, the kidney.

### Fatty Acid Mixture

Fatty acid mixtures contain fatty acids, including eicosapentaenoic acid (EPA) and docosahexaenoic acid (DHA). Preferred fatty acids are essential omega-3 fatty acids. The omega-3 fatty acids can derive from small feeder fish typically found at or near the bottom of the food chain, including sardines, anchovies, and mackerel. These marine species are advantageously devoid of the contaminants typically associated with more predatory, higher marine species.

Molecularly distilled fish body oil that is highly purified, concentrated and standardized can provide specific amounts of essential omega-3 (n-3) poly-unsaturated fatty acids (PUFAs), including docosahexaenoic Acid (DHA) and eicosapentaenoic Acid (EPA).

Table 5A shows the composition range of components for useful fatty acid mixtures for use with radiation and oxidative exposure treatment compositions. Table 5B shows the daily dose of a useful mixture of fatty acids for use with radiation and oxidative exposure treatment compositions.

TABLE 5A
Composition range for daily doses of the components
for useful fatty acid mixtures for use with
radiation and oxidative exposure treatment compositions

| | Daily Dose | Units of |
|---|---|---|
| Ingredient | Range | Measure |
| Eicosapentaenoic Acid (EPA) | 0-2000 | mg |
| Docosahexaenoic Acid (DHA) | 0-2000 | mg |

In some embodiment mixtures, the total amount of omega-3 fatty acids in the fatty acid mixture is about 1200 mg.

TABLE 5B
Daily dose of a useful mixture of fatty acids
for use with radiation and oxidative
exposure treatment compositions

| | | Units of |
|---|---|---|
| Ingredient | Daily Dose | Measure |
| DHA (from algal oil and/or fish oil) | 720 | mg |
| EPA (from fish oil) | 1080 | mg |

In some embodiment mixtures of fatty acids, the amount of EPA for a daily does is about 720 mg. In some other embodiment mixtures of fatty acids, the amount of DHA for a daily does is about 480 mg.

Following ingestion, EPA and DHA undergo hydrolysis via lipases to form monoglycerides and free fatty acids. In the enterocytes, reacylation takes place and this results in the formation of triacylglycerols, which are then assembled with phospholipids, cholesterol and apoproteins into chylomicrons. These are then released into the lymphatic system from whence they are transported to the systemic circulation. Here, the chylomicrons are degraded by lipoprotein lipase, and EPA & DHA are transported to various tissues of the body via blood vessels, where they are used mainly for the synthesis of phospholipids. Phospholipids are incorporated into the cell membranes of red blood cells, platelets, neurons and others. EPA and DHA are mainly found in the phospholipid components of cell membranes. DHA is taken up by the brain and retina in preference to other fatty acids. DHA can be partially and conditionally reconverted into EPA, and vice-versa, although the process is thought to be less-than-efficient and may be adversely affected by age.

Eicosapentaenoic acid (EPA) and docosahexaenoic acid (DHA) are essential omega-3 fatty acids and both play a role in the formation of anti-inflammatory and immunomodulating eicosanoids. As such, they have several actions in a number of body systems. Both play an important role in the maintenance of normal blood flow as they lower fibrinogen levels. DHA is vital for normal neurological function throughout life. Several mechanisms are believed to account for the anti-inflammatory activity of EPA and DHA. Most notably, the two competitively inhibit the conversion of arachidonic acid to the pro-inflammatory prostaglandin E2 (PGE2), and leukotriene B4 (LKB4), thus reducing their synthesis. EPA and DHA also inhibit the synthesis of the inflammatory cytokines Tumor Necrosis Factor-alpha (TNF-a), Interleukin-1 (IL-1) beta. EPA and DHA inhibit the 5-LOX (lipoxygenase) pathway responsible for the conversion of arachidonic acid to inflammatory leukotrienes in neutrophils and monocytes and can suppress phospholipase C-mediated signal transduction, also involved in inflammatory events. EPA is the precursor to series-3 prostaglandins, series-5 leukotrienes (LTBS) and series-3 thromboxanes (TXA3). This could account in part for its microvascular and anti-inflammatory role. Furthermore, EPA is a precursor of resolvins (Rv) such as RvE1 and RvD1 which may help reduce tear gland inflammation, increase tear volume and ocular lubrication.

EPA and DHA have both similar and dissimilar physiologic roles. EPA appears to be more important in those roles where the eicosanoids are involved such as inflammation as well as tear gland function and tear production, whereas DHA seems to play its most important role in offering structural protection to the retina and other neurovascular structures such as corneal nerves.

### Additional Formulations:

Table 6 shows the composition range of components for useful micronutrient multivitamin and trace element mixtures for use with the daily dose radiation and oxidative exposure treatment compositions.

TABLE 6
Composition range for daily doses of useful
micronutrient multivitamin and trace
element mixtures for use with radiation and oxidative
exposure treatment compositions.

| | Daily Dose | |
|---|---|---|
| | Range | Units of |
| Ingredient | (Label Claim) | Measure |
| Total Vitamin A | 3125-6,167 | IU |
| Vitamin C | 60-150 | mg |
| Vitamin D | 400-1,780 | IU |
| Vitamin E | 30-100 | IU |
| Vitamin K | 45-80 | mg |
| Thiamine (Vitamin B1) | 1.5-23.6 | mg |
| Riboflavin (Vitamin B2) | 1.7-10.4 | mg |
| Niacin (as inositol hexanicotinate, | 20-50 | mg |
| niacin or niacinamide) | | |
| Vitamin B6 | 2-43.6 | mg |
| Folate | 200-1,060 | μg |
| Vitamin B12 | 6-57.6 | μg |
| Biotin | 187-450 | μg |
| Pantothenic acid | 10-25 | mg |
| Calcium | 0-1,200 | mg |
| Iodine | 15-2,070 | μg |
| Magnesium | 0-400 | mg |
| Zinc | 15-23 | mg |
| Selenium | 70-190 | μg |
| Copper | 0-5 | mg |
| Manganese | 1.25-7 | mg |
| Chromium | 0-200 | μg |
| Molybdenum | 0-100 | μg |
| Alpha Lipoic Acid | 100-1,000 | mg |
| Citrus Bioflavonoids (e.g. quercetin) | 50-100 | mg |
| Pine Bark Extract | 20-50 | mg |
| Lycopene | 1-10 | mg |
| Lutein | 1-15 | mg |
| Astaxanthin | 0.25-10 | mg |

Blending in suitable devices combines the components of each mixture. For example, mixing can occur in a V-type blender. One of ordinary skill in the art can determine the devices and apparatuses best suited for combining the components of the mixture comprising nonessential natural antioxidants and chemoprevention agents.

Radiation-oxidative exposure treatment compositions, which comprise micronutrient vitamins, trace elements, non-essential natural antioxidants, chemoprevention agents and optionally fatty acids, can ameliorate the chronic, life-shortening effects of radiation exposure after exposure. Treatment with radiation-oxidative exposure treatment compositions can also ameliorate organ-specific late radiation injuries, which may include pulmonary fibrosis, renal failure, hepatic fibrosis and central nervous system damage, which can result in neuro-cognitive impairment. As well, treatment with radiation-oxidative exposure treatment compositions can also ameliorate the acute effects of total-body irradiation.

### Administration of the Radiation-Oxidative Exposure Treatment Composition

The radiation-oxidative exposure treatment compositions as described, which contain amounts of micronutrient multivitamin, trace elements, non-essential antioxidants, chemopreventative agents, and optionally fatty acids, are useful for pre- or post-exposure treatment to radiation sources or sources of oxidative stress, or both, that impact a subject. Exposure to either or both of these damaging sources can induce life-shortening effects. Daily administration of the radiation-oxidative exposure treatment compositions can ameliorate these post-exposure life-shortening effects. The composition can be effective for subjects exposed to radiation in outer space.

The administration of the radiation-oxidative exposure treatment compositions can be self-introduced, making oneself the subject of the daily administration of the treatment. Examples of self-introduction include orally consuming the composition with meals or as capsules, injecting oneself with a solution comprising the composition, and applying an ointment comprising the composition to one's skin. Examples of administration of the radiation-oxidative exposure treatment compositions to a subject not oneself include feeding a subject a foodstuff comprising the composition as part of a daily meal and injecting a subject with a solution comprising the composition. One of ordinary skill in the art can device numerous methods of administering radiation-oxidative exposure treatment compositions to various subjects to effect the proper daily dose. These can include time-release capsules, orally ingested liquids, intraperitoneal, intravenous, subcutaneous, sublingual, transcutaneous, intramuscular, and other well-understood forms.

“Subjects” include, without limitation, animals, which include mammals, which also include dogs, cats, mice and humans (Homo sapiens).

The radiation-oxidative exposure treatment compositions are “daily dose” amounts. That is, the radiation-oxidative exposure treatment compositions as described represent the amount of radiation-oxidative exposure treatment compositions that are for administration during a 24-hour period or on a daily basis to a subject to ameliorate the life shortening effects of radiation exposure or oxidative stress, or both.

The radiation-oxidative exposure treatment composition can be administered or introduced to a subject as a pure or refined material. Typically, the composition is dilution by blending with other materials for ingestion or injection, including foodstuffs (water, drinks, meals, chow mixes) edible solids, gels; palatable liquids and solutions; salines and fluids for intramuscular administration; and inert binding materials.

Oral consumption is the preferred method of administration since digestion metabolizes many of the component mixtures, especially antioxidant compounds, into their active and protective forms. Oral consumption is also a comfortable and palatable delivery vehicle for introduction of the radiation-oxidative exposure treatment compositions versus more invasive means. Forms of the radiation-oxidative exposure treatment composition for oral administration, either in pure or diluted form, include lacquered or coated tablets, unlacquered or uncoated tablets, caplets, hard capsules, liquid-filled capsules, hard gelatin capsule, hard vegetable-based capsule, elixir, soft-chew, lozenge, chewable bar, juice suspension, liquids, time-release formulations, and foodstuffs.

The daily dosage can be administered in the form of one or more capsules. The formulation of an individual capsule is determined based on the amount of the essential ingredients that are required to be present in each capsule to total the amount of essential ingredients. For simplicity, during the remaining portion of this description, the form of administration, whether lacquered tablets, unlacquered tablets, caplets, or capsules, will be referred to as “capsules” without distinguishing among the various forms.

An example foodstuff that includes a daily dose of the radiation-oxidative exposure treatment composition for oral administration comprises 0.024% of the micronutrient multivitamin and trace elements by total weight of the foodstuff and 0.023% of the antioxidant and chemopreventative agent mixture by total weight of the foodstuff, with the remainder of the foodstuff used for blending down the radiation-oxidative exposure treatment composition.

If a foodstuff or other material for oral consumption is used for administering the radiation-oxidative exposure treatment composition, it is preferable that components of the foodstuff or other materials do not react with, interfere with the processing or absorption of, or negate the desirable properties of the radiation-oxidative exposure treatment composition.

The entire daily dose of the radiation-oxidative exposure treatment composition does not have to be administered in a single dose during a 24-hour period. The radiation-oxidative exposure treatment composition sub-divided and proportionally administered more than once per day. The daily dose appropriately apportioned reflects the number of administrations to occur during the day. For example, it may be easier to administer the daily dose of radiation-oxidative exposure treatment composition as three, one-third portions three times a day. In this example, tri-daily consumption of one-third portions of the radiation-oxidative exposure treatment composition can occur with three regularly scheduled meals and effects the daily dose for the subject. Dividing the daily dose into smaller, more frequent administrations can improve the habit of self-administration, make it easier to audit to determine if proper dosage has occurred, and make the consumption of the radiation-oxidative exposure treatment composition more tolerable to those with highly-sensitive taste. The sum of the proportional amounts of the administered composition during the 24-hour period should total the daily dose of the composition.

The radiation-oxidative exposure treatment composition mixtures can be administered separately to effect the proper daily dose of the radiation-oxidative exposure treatment composition. For example, the antioxidant and chemopreventative agent mixture can be provided for in separate capsules from the fatty acid mixture and the micronutrient multivitamin and trace element mixture. In another example, the antioxidant and chemopreventative agent mixture and the micronutrient multivitamin and trace elements mixture can be compounded together and the fatty acid mixture provided as a separate mixture. One of ordinary skill in the art can devise a variety of dosage schedules and partitions of the mixtures comprising the radiation-oxidative exposure treatment composition to effect the proper administration of the daily dose.

The radiation-oxidative exposure treatment composition mixtures can be sub-divided and proportionally administered during a 24-hour period to effect the proper daily dose of the radiation-oxidative exposure treatment composition. For example, the daily dose of the radiation-oxidative exposure treatment compositions can be administered through three capsules of a micronutrient multivitamin and trace elements, each capsule containing a third of the daily dose of the micronutrient multivitamin and trace elements mixture; three capsules of antioxidants and chemopreventative agents, each capsule containing a third of the daily dose of the antioxidant and chemopreventative agents mixture; and two soft liquid-filled capsules containing fatty acids, each containing half of the daily dose of the fatty acids. One of ordinary skill in the art can devise a variety of dosage schedules and partitions of the radiation-oxidative exposure treatment composition mixtures to effect the proper administration of the daily dose. The sum of the proportional amounts of the administered mixture during the 24-hour period should total the daily dose of the mixture, and the sum of the proportional amounts of radiation-oxidative exposure treatment composition should total the daily dose for the composition.

Research suggests that fat soluble antioxidants such as carotenoid lutein are best absorbed when combined with fat (e.g., oils). The fatty acid mixture comprises molecularly distilled fish oil as a source of omega-3 fatty acids, which also acts as a carrier and solubilizer for these carotenoids. This reduces the need to take the capsules with a fatty meal. Nevertheless, it is believed that combining the dose with the intake of a small meal containing a healthy portion of fat (i.e., olive oil, salmon, etc) may further help in the proper assimilation of the active components. It is preferable to avoid taking at the same time as foods rich in oxalic or phytic acid (e.g., raw beans, seeds, grains, soy, spinach, rhubarb), as they may depress the absorption of minerals like zinc; however, it is not necessary to avoid these foods for the composition to still be effective.

A delayed-release mechanism through enteric coating of soft liquid-filled capsules can be provided. Such a coating helps to reduce gastroesophageal reflux and fishy odor. The capsule can be coated in order to enhance the bioavailability of the dosage by maintaining the integrity of the fatty acids, minimizing their exposure to the gastric environment, and maximizing the capsule's disintegration upon its arrival at the duodenum.

The active ingredients of radiation-oxidative exposure treatment composition may be presented in a variety of forms. Additionally, the method of manufacturing may take a variety of forms and a number of inactive ingredients may be added to provide longer shelf life, to make the capsule more palatable or presentable, and to aid in the ease of manufacturing process. The capsules may be blended with any desired inactive ingredients, so long as the blend is uniform and the appropriate composition is reached for each capsule. The capsules may be coated or they can be contained in a carrier, such as mineral oil, to produce a soft gel.

The actual capsules containing parts or all of the radiation-oxidative exposure treatment composition mixtures may contain somewhat more than the total amounts specified as the daily dose since the active ingredients may degrade over time. Consequently, in order to assure that the active ingredients are present in the minimum amounts required at the time the capsules are actually ingested, may require increasing the dosage beyond the minimum amounts required in order to account for and compensate for degradation over time. Some of the essential ingredients degrade faster than others, which can result in different percentages of excess in each capsule for one essential ingredient as compared to a different essential ingredient.

Prior animal-based studies also show that 7-10 days of oral administration of diets rich in antioxidants result in significant elevations in levels of micronutrients. Although not intending to be bound by theory, it is believed that administering radiation-oxidative exposure treatment compositions on a continuing daily basis for at least 7-10 days before exposure to a radiation source maximizes the concentration of beneficial components for radiation exposure treatment in the subject at the time of radiation exposure.

Animal-based studies also suggest that administration of combinations of vitamins, trace elements, non-essential natural antioxidants, and chemopreventative agents during and after exposure to a radiation source provides a source of continual antioxidant bioavailability that improves both acute as well as long-term survival due to the reduction in radiation-induced life shortening caused by total-body irradiation. Although not intending to be bound by theory, it is believed that this effect also works for oxidative stress-induced damage. The period for continuing daily administration of the daily dose of radiation-oxidative exposure treatment compositions can be in a range of from about 1 day after exposure to the end of the subject's lifespan. The experiment shows beneficial administration of a radiation-oxidative exposure treatment composition for up to 450 days.

Experimental models demonstrate the use of radiation-oxidative exposure treatment compositions in ameliorating the acute effects of radiation. These models show strongly imply that the long-term effects are transferable to other animal species, including other mammals, and especially to humans (Homo sapiens).

Methods of pre- or post-exposure treatment can include the additional step of administering manganese superoxide dismutase plasmid DNA in liposome (MnSOD-PL) gene product intravenously in conjunction with receiving daily doses of radiation-oxidative exposure treatment compositions. The additional step can further decrease radiation-induced cellular apoptosis, tissue injury, and improve the survival rate in organ-specific and total-body-irradiated rodents.

Administration of a MnSOD-PL injection at least 24 hours prior to total-body irradiation not only improves survival from the LD50 dose of 9.5 Gy in C57BL/6HNsd mice but also ameliorates the late radiation-induced life shortening in male mice. Radiation-oxidative exposure treatment compositions also improves the long-term survival rate in acutely irradiated mice by reducing radiation-induced life shortening effects.

Intravenous injection of MnSOD-PL (at a dilution of 100 μg of plasmid DNA to 100 μL of liposomes) gene product at least about 24 hours before irradiation can provide some protective benefit. The injection amount is about 0.004 grams plasmid DNA per kilogram subject bodyweight.

Test mice receiving a MnSOD-PL injection prior to irradiation and demonstrating improved survival after the LD50/30 dose also show amelioration of radiation-induced late effects. Although not intending to be bound by theory, it is believed that these results are attributable to a decrease in radiation-induced aging in a non-specific sense rather than a decrease in the frequency or type of radiation-induced tumors or evidence of neurodegenerative disease. Since radiation-induced life shortening associates with biomarkers of aging, including fur graying in rodent models, organ failure, osteoporosis and fibrosis, many animals in these prior studies do not show specific causes of death. Additionally, prior studies indicate antioxidant MnSOD-PL treatment does not increase tumor frequency or lethality.

Examples of specific embodiments facilitate a better understanding of radiation-oxidative exposure treatment compositions and their use in ameliorating radiation-induced life shortening effects after exposure to a radiation source. In no way should the Examples limit or define the scope of the invention.

### Experiment

### Mice and Animal Care

The mammal models are 160 female C57BL/6NHsd mice, aged 8 weeks. There are four groups of 40 mice each. Each mouse weighs approximately 22.5 grams.

The University of Pittsburgh Institutional Animal Care and Use Committee approves all experimental protocols. The University of Pittsburgh Division of Laboratory Animal Research provides veterinary care. The model animals are C57BL/6HNsd female mice. Each cage houses five mice during the study. Maintenance and housing of the mice occurs according to the protocols of The University of Pittsburgh Division of Laboratory Animal Research.

### Experimental Protocols

For the experiment, an “experimental” chow mix with dietary supplements sustains two of the four groups of 40 mice. The diet of chow mix in combination with the dietary supplement sustains these two groups from 7 days before the before irradiation until conclusion of the experiment. A “house” chow mix maintains the other two groups of 40 mice for the same period for control purposes. The chow portion per mouse per day is 5,000 mg.

The base chow mix is “Lab Diet rMH 3000 (5P00)” (Cat. No. 1812877) from TESTDIET (Richmond, Ind.).

The house chow mix comprises 0.12% hydrogen silicon dioxide by total weight of the house chow mix and the remainder is base chow mix. The silicon dioxide, which is inert and not harmful to the mice, compensates for any potential changes in the weight of the mice due to the addition of the dietary supplement.

Table 7 shows the constituents of both the first dietary supplement mixture comprising micronutrient vitamins and trace elements and the second dietary supplement mixture comprising non-essential natural antioxidants and chemoprevention agents. AmeriSciences LP (Houston, Tex.) supplies the first dietary supplement mixture as “AmeriSciences/NASA Premium Multivitamin Premix”. AmeriSciences LP also supplies the second dietary supplement mixture as “AmeriSciences/NASA Fruit and Veggie Antioxidant Formula Premix”.

Units of measure for Tables 7 includes “IU”, which represents “International Units”, an understood metric in the art for measuring the active amount of particular species, especially vitamins (e.g., Vitamins A, D, and E). Milligrams (“mg”) are 1×10-3 grams. Micrograms (“μg”) are 1×10-6 grams.

Table 7 also shows dietary supplement mixture amounts for both model mice (˜22.5 grams) and the equivalent human daily dose for the two dietary supplement mixtures. The table also provides information regarding Human UL (“tolerable upper intake level”) and Human NOAFL (“no observed adverse effect level”) levels for the micronutrient vitamins and trace elements mixture.

TABLE 7
Dietary supplements containing micronutrient vitamins and trace elements and
nonessential natural antioxidants and chemoprevention agents.

| | | | Equivalent | | Human UL* | | | |
|---|---|---|---|---|---|---|---|---|
| | Daily dose | | Human | | (19-70 | | Human | |
| MICRONUTRIENT COMPONENTS: | per mouse† | | Daily Dose | | yo group) | | NOAEL** | |
| Vitamin A (30% as vitamin A palmitate and | 0.2451 | IU | 750 | IU | 10,000 | U | 10,000 | IU |
| 70% as beta-carotene) | | | | | | | | |
| Beta-carotene (part of Vitamin A total) | 0.3431 | mcg | 1.05 | mg | NE‡ | | 25 | mg |
| Vitamin C (as ascorbic acid) | 0.0817 | mg | 250 | mg | 2000 | mg | >1000 | mg |
| Vitamin D (as cholecalciferol) | 0.3921 | IU | 1200 | IU | 4000 | IU | 800 | IU |
| Vitamin E (as d-alpha tocopheryl succinate and | 0.0653 | U | 200 | IU | 1490 | IU | 1200 | IU |
| mixed tocopherols) | | | | | | | | |
| Vitamin K (as phytonadione) | 0.0261 | mcg | 80 | mcg | NE | | 30 | mcg |
| Thiamine (vitamin B1) (as thiamine mononitrate) | 0.7352 | mcg | 2.25 | mg | NE | | 50 | mg |
| Riboflavin (vitamin B2) | 0.8332 | mcg | 2.55 | mg | NE | | 200 | mg |
| Niacin (as inositol hexanicotinate) | 9.802 | mcg | 30 | mg | 35 | mg | 500 | mg |
| Vitamin B6 (as pyridoxine hydrochloride) | 0.9802 | mcg | 3 | mg | 100 | mg | 200 | mg |
| Folate (as folic acid) | 0.1960 | mcg | 600 | mcg | 1000 | mcg | 1000 | mcg |
| Vitamin B12 (as cyanocobalamin) | 0.0029 | mcg | 9 | mcg | NE | | 3000 | mcg |
| Biotin | 0.1470 | mcg | 450 | mcg | NE | | 2500 | mcg |
| Pantothenic acid (as d-calcium pantothenate) | 4.901 | mcg | 15 | mg | NE | | 1000 | mg |
| Calcium (as calcium carbonate, dicalcium phosphate) | 0.1634 | mg | 500 | mg | 2500 | mg | 1500 | mg |
| Iodine (from kelp) | 0.0098 | mcg | 30 | mcg | 1100 | mcg | 1000 | mcg |
| Magnesium (as magnesium oxide and chelate) | 65.35 | mcg | 200 | mg | 350 | mg | 700 | mg |
| Zinc (as zinc chelate [monomethionine]) | 4.901 | mcg | 15 | mg | 40 | mg | 30 | mg |
| Selenium (as L-selenomethionine) | 0.0327 | mcg | 100 | mcg | 400 | mcg | 200 | mcg |
| Copper (as copper amino acid chelate) | 0.0588 | mcg | 0.18 | mg | 10 | mg | 9 | mg |
| Manganese (as manganese amino acid chelate) | 0.6535 | mcg | 2 | mg | 11 | mg | 10 | mg |
| Chromium (as chromium polynicotinate) | 0.0653 | mcg | 200 | mcg | NE | | 1000 | mcg |
| Molybdenum (as molybdenum amino acid chelate) | 0.0183 | mcg | 56 | mcg | 2000 | mcg | 350 | mcg |

| Potassium (as potassium citrate) | 94.75 | mcg | 290 | mg | NE | | NE |
|---|---|---|---|---|---|---|---|
| Choline (as choline bitartrate) | 16.34 | mcg | 50 | mg | 3500 | mg | NE |
| Inositol (as inositol and inositol hexanicotinate) | 16.34 | mcg | 50 | mg | NE | | NE |
| Boron (as boron chelate) | 0.3267 | mcg | 1 | mg | 20 | mg | NE |
| Vanadium (as vanadyl sulfate) | 0.0163 | mcg | 50 | mcg | 1800 | mcg | NE |

| NON-ESSENTIAL NATURAL ANTIOXIDANT | | | | |
|---|---|---|---|---|
| & CHEMOPREVENTION AGENTS: | | | | |
| Rutin | 8.036 | mcg | 25 | mg |
| Quercetin | 257.1 | mcg | 800 | mg |
| Hesperidin | 1.607 | mcg | 5 | mg |
| Alpha Lipoic Acid | 128.6 | mcg | 400 | mg |
| N-Acetyl-L-Cysteine (NAC) | 192.9 | mcg | 600 | mg |
| Lutein | 3.214 | mcg | 10 | mg |
| Lycopene | 1.607 | mcg | 5 | mg |
| Astaxanthin | 0.3214 | mcg | 1 | mg |
| Plant Sterols | 80.36 | mcg | 250 | mg |
| Isoflavones (from soy extract) | 8.036 | mcg | 25 | mg |
| Garlic Extract (bulb) | 88.39 | mcg | 275 | mg |
| Green Tea Extract (leaf) | 80.36 | mcg | 250 | mg |
| [standardized to 95% polyphenols and 50% | | | | |
| epigallocatechin gallate (EGCG)] | | | | |
| Cruciferous Vegetable Extract (Brassica spp.) (plant) | 32.14 | mcg | 100 | mg |
| Fruit Blend | 32.14 | mcg | 100 | mg |
| (strawberry, escobillo, blueberry, blackberry, | | | | |
| cranberry, grape, pomegranate) | | | | |
| Ginkgo Biloba Extract (leaf) | 19.29 | mcg | 60 | mg |
| Coenzyme Q-10 | 32.14 | mcg | 100 | mg |
| Resveratrol | 1.607 | mcg | 5 | mg |

†Note:
Each mouse weighs an average of 22.5 grams.
*UL: Dietary Intakes' “Tolerable Upper Intake Levels.” The maximum level of daily nutrient intake that is likely to pose no risk of adverse effects. Food and Nutrition Board, Institute of Medicine, National Academy of Science.
**NOAEL: “No Observed Adverse Event Level” is a level that should be considered safe and requires no application of a safety factor to determine a safe intake, based on the most sensitive subgroup.
‡NE: None Established

The experimental chow mix that sustains the other two groups of 40 mice includes both the first and second dietary supplement mixtures with the base chow mix. The experimental chow mix comprises 0.024% “AmeriSciences/NASA Premium Multivitamin Formula” by total weight of the experimental chow mix, 0.023% “AmeriSciences/NASA Fruit/Veggie Antioxidant Formula” by total weight of the experimental chow mix, and the remainder base chow mix. The experimental chow mix contains 1.22 mg per day of AmeriSciences/NASA Premium Multivitamin Formula and 1.13 mg per day of AmeriSciences/NASA Fruit/Veggie Antioxidant Formula. Based upon an average weight per mouse of 22.5 grams, each mouse ingests at a rate of 0.05 grams of AmeriSciences/NASA Premium Multivitamin Formula per kilogram subject bodyweight per day and 0.05 grams of AmeriSciences/NASA Fruit/Veggie Antioxidant Formula per kilogram subject bodyweight per day.

There are no other additional ingredients for either the house chow or the experimental chow mixes. The Purina Corporation combines all the additives and forms both chow mixes into feed pellets of similar size and shape.

Intravenous injection of MnSOD-PL (100 μg of plasmid DNA in 100 μL of liposomes) gene product occurs about 24 hours before irradiation into one of the two experimental chow mix diet groups (40 mice) and into one of the two house chow mix diet groups (40 mice) according to methods known in the art. Given the average weight of a mouse in the experiment is 22.5 grams, the injection amount is about 0.004 grams plasmid DNA per kilogram subject bodyweight. The feed schedule and mixes for both groups remains unchanged.

A J. L. Shepherd Mark I cesium irradiator exposes all models to a 9.5 Gy total-body radiation dose at a rate of 70 cGy/min 24 hours after the two MnSOD-PL injected mice receive their injections and after 7 days of feeding with either the house or experimental chow mixes. “Gy” is a gray, which is the absorption of one joule of ionizing radiation by one kilogram of matter.

### Statistical Evaluation of Experimental Models

Evaluations of the models are for survival, overall survival and conditional survival. “Overall survival” is the time from the date of irradiation to the date of expiration for any model under study. “Conditional survival” is the time from the date of irradiation to the date of expiration for all mice that survive 31 days or longer after irradiation.

The two-sided Fisher's exact test compares model 30-day mortality between any two different diet and injection status groups. The two-sided log-rank test compares two different diet and injection status groups having models surviving 31 days or longer. Comparative P-values of less than 0.050 are significant. SAS software (SAS Institute, Inc; Cary, N.C.) provides statistical analysis and computational results for the studies.

### Results

Mice on the house chow diet compared to experimental chow diet did not show any differences in body weight over the 450-day post-irradiation period. This indicates that the experimental chow diet containing the micronutrient vitamins, trace element, non-essential natural antioxidants and chemoprevention agent diet is similarly palatable to the mice as the house chow.

Table 8 provides statistical analysis information regarding 30-day mortality and average survival rates for the models surviving more than 30 days after exposure to the acute radiation source for each of the four groups and comparatively.

TABLE 8
Thirty day and long-term mortality rates after 9.5 Gy total body irradiation of mice in
relation to experimental chow mix diet and injection of MnSOD-PL gene product versus house
mix diet.

| | | | | Survival > 30 days* | |
|---|---|---|---|---|---|
| | | 30 day mortality | | Median | |
| Group | n | % | P** | (95% CI) | P^{†} |
| 1. Control | 40 | 45 | | 213 | |
| | | | | (161-291) | |
| 2. MnSOD-PL | 40 | 20 | 0.031 | 328 | 0.020 |
| | | | (compared to group 1) | (216-373) | (compared to group 1) |
| 3. Anti-oxidant diet | 40 | 50 | 0.823 | 309.5 | 0.040 |
| | | | (compared to group 1) | (231-373) | (compared to group 1) |
| 4. Anti-oxidant diet + | 40 | 17.5 | 0.015 | 322 | 0.010 |
| MnSOD-PL | | | (compared to group 1) | (287-358) | (compared to group 1) |
| | | | 1.000 | | 0.953 |
| | | | (compared to group 2) | | (compared to group 2) |
| | | | 0.004 | | 0.874 |
| | | | (compared to group 3) | | (compared to group 3) |

*Analysis for animals surviving more than 30 days
**Fisher's exact test
^{†}Log-rank test

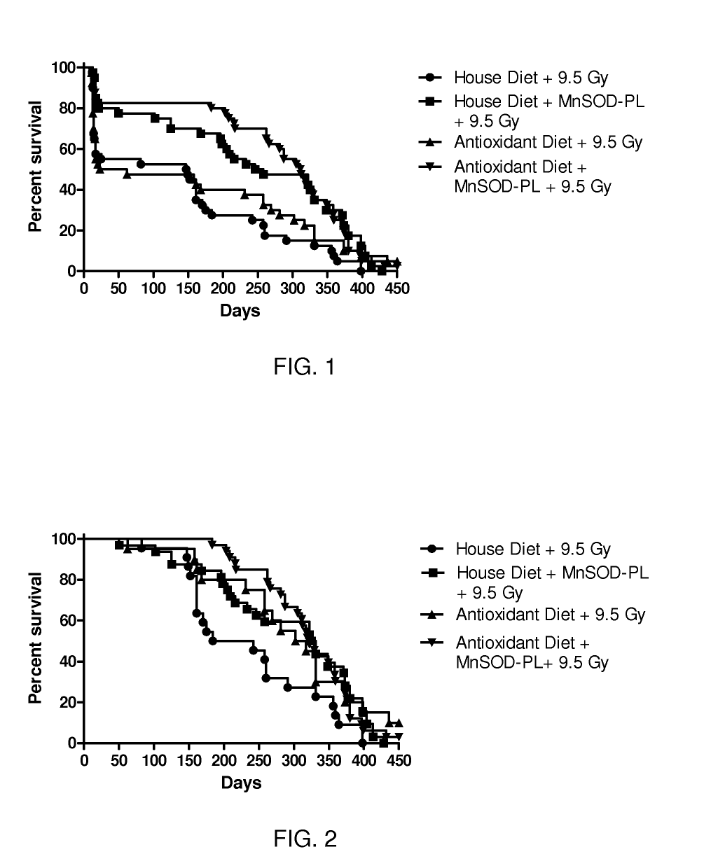

FIGS. 1 and 2 and their description facilitate a better understanding of overall survival and conditional survival for the members of the four model groups in the experiment. In no way should either FIG. 1 or 2 limit or define the scope of the invention.

FIG. 1 is a graph showing percentage overall survival of the members of four model groups receiving 9.5 Gy of radiation for the period of 450 days after initial exposure. FIG. 2 is a graph showing percentage condition survival of the members of the four model groups after receiving 9.5 Gy of radiation during the period of 30 days from initial exposure to 450 days after initial exposure.

MnSOD-PL Administration Improves Survival after LD50/30 Total-Body Irradiation

Table 8 indicates that mice receiving intravenous administration of MnSOD-PL gene product show improved survival compared to mice in the control group (house chow diet) after 9.5 Gy TBI exposure. The data in Table 8 confirms and demonstrates decreased 30-day mortality in the MnSOD-PL gene product injection/house chow group compared to the no injection/house chow control: 20% mortality in the MnSOD-PL group compared 45% in the control (P=0.031). FIG. 1 also shows this increased survival rate from the acute exposure.

Table 8 shows mice receiving the no injection/experimental chow diet did not show an improvement in survival up to the thirty day mark, having a mortality of 50%, compared to 45% for the no injection/house chow control (P=0.82).

Thirty-day mortality is significantly lower in MnSOD-PL gene product injection/experimental chow group compared to the no injection/house chow control and no injection/experimental chow diet: 17.5% for the antioxidant diet+MnSOD-PL group compared to 45% mortality in no injection/house chow control and 50% in the no injection/experimental chow diet (P=0.015 and 0.004, respectively). These results establish that the experimental chow, which contains the first and second dietary supplement mixtures, does not negatively affect the radio-protective effect of MnSOD-PL gene product against total-body irradiation.

### Antioxidant Diet Improves Conditional Survival and Ameliorates Radiation-Induced Life Shortening

Evaluation for late effects of radiation (conditional survival) occurs for mice surviving beyond 30 days after irradiation. FIG. 2 and Table 8 shows that the conditional survival of mice on the experimental chow diet significantly improves over the remainder of the 450 days of observation period compared to that of those on the house chow diet control group (P=0.040). Mice on the house chow diet also receiving the MnSOD-PL gene product injection show improvement in conditional survival rates compared to the house chow diet control group with no injection (P=0.020). The MnSOD-PL gene product injection/experimental chow group also show improvement in conditional survival compared to the no injection/house chow diet control (P=0.010). There is no significant difference in conditional survival between the MnSOD-PL gene product injection/experimental chow group and both the MnSOD-PL gene product injection/house chow group or no injection/experimental chow diet group.

Among the irradiated mice surviving 31 days or longer, Table 8 shows the conditional median survival time is 213 days for the no injection/house chow diet controls, 328 days for the MnSOD-PL gene product injection/house chow group, 309.5 days for the no injection/experimental chow group, and 322 days for the MnSOD-PL gene product injection/experimental chow group.

The conditional survival results establish that the supplement mixture comprising micronutrient vitamins and trace elements and the supplement mixture comprising non-essential natural antioxidants and chemoprevention agents ameliorate radiation-induced life shortening. The results support the concept of abating continuing oxidative stress in the post-irradiation cellular microenvironment of tissues, organs and organ systems with mixtures of micronutrient vitamins and trace elements, non-essential natural antioxidants and chemoprevention agents.

The experiment shows the composition comprising the micronutrient vitamins, trace elements, non-essential natural antioxidants and chemoprevention agents improves conditional survival in total-body-irradiated female mice. A significant therapeutic effect of the experimental chow diet is in conditional survival. In animals surviving the acute effects of radiation, the diet containing the micronutrient vitamins, trace elements, non-essential natural antioxidants and chemoprevention agents ameliorates radiation-induced life shortening.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used have the same meaning as commonly understood by one of ordinary skill in the art to which this invention belongs. Although any methods and materials similar or equivalent to those described can also be used in the practice or testing of the invention, a limited number of the exemplary methods and materials are described.

As used in the description and in the appended claims, the singular forms “a”, “an”, and “the” include plural referents unless the context clearly dictates otherwise.

All publications mentioned are incorporated by reference to disclose and describe the methods or materials, or both, in connection with which the publications are cited. The publications discussed are provided solely for their disclosure prior to the filing date of the present application. Nothing is to be construed as an admission that the invention is not entitled to antedate such publication by virtue of prior invention. Further, the dates of publication provided may be different from the actual publication dates, which may need to be independently confirmed.

It should be apparent to those skilled in the art that many more modifications besides those already described are possible without departing from the inventive concepts. The inventive subject matter, therefore, is not restricted except in the spirit of the disclosure. By way of example, and without limitation, the tested agents described herein can be provided in an isolated form, or can be provided via another substance that contains the agent, for example, yeast as a glutathione-containing substance.

In interpreting the disclosure, all terms should be interpreted in the broadest possible manner consistent with the context. In particular, the terms “comprises” and “comprising” should be interpreted as referring to elements, components, or steps in a nonexclusive manner, indicating that the referenced elements, components, or steps may be present, or utilized, or combined with other elements, components, or steps that are not expressly referenced.

Where reference is made to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

What is claimed is:

1. A radiation-oxidative exposure treatment composition for ameliorating radiation-induced life shortening effects from exposure to a radiation source and the effects of oxidative stress such as premature skin aging due to sun exposure, exercise-induced muscle fatigue, oxidative stress of the liver due to toxic exposures, reduction in visual acuity, the radiation-oxidative exposure treatment composition comprising:
a) a mixture of micronutrient multivitamin and trace elements,
b) a mixture of antioxidants and chemopreventative agents, and
c) optionally, a mixture of fatty acids;
wherein the mixture of micronutrient multivitamin and trace elements comprises:
an amount of biotin in a range of from about 0.187 to about 0.45 mg;
an amount of folate in a range of from about 0.25 to about 1.06 mg;
an amount of iodine in a range of from about 0.0187 to about 2.07 mg;
an amount of manganese in a range of from about 1.25 to about 7 mg;
an amount of pantothenic acid in a range of from about 10 to about 25 mg;
an amount of selenium in a range of from about 0.0875 to about 2.07 mg;
an amount of vitamin B1 in a range of from about 1.5 to about 23.6 mg;
an amount of vitamin B12 in a range of from about 0.0075 to about 0.009 mg;
an amount of vitamin B2 in a range of from about 1.7 to about 10.4 mg;
an amount of vitamin B3 in a range of from about 25 to about 50 mg;
an amount of vitamin B6 in a range of from about 2 to about 43.6 mg;
an amount of vitamin C in a range of from about 60 to about 500 mg;
an amount of vitamin D in a range of from about 0.0125 to about 0.0445 mg;
an amount of vitamin E in a range of from about 20 to about 643 mg;
an amount of vitamin K in a range of from about 0.056 to about 0.080 mg;
an amount of lycopene in a range of from about 1 to about 5 mg;
an amount of zinc in a range of from about 15 to about 40 mg;
an amount of alpha lipoic acid in a range of from about 100 to about 125 mg;
an amount of astaxanthin in a range of from about 0.25 to about 0.31 mg;
an amount of lutein in a range of from about 1 to about 6.25 mg;
an amount of vitamin A in a range of from about 0.90 to about 1.85 mg;
an amount of quercetin in a range of from about 0 to about 64 mg;
an amount of glutathione in a range from about 0 to about 125 mg;
an amount of pine bark extract in a range from about 0 to about 50 mg;
an amount of curcumin extract in a range from about 0 to about 250 mg;
an amount of calcium in a range of from about 0 to about 1,200 mg;
an amount of chromium in a range of from about 0 to about 0.2 mg;
an amount of copper in a range of from about 0 to about 5 mg;
an amount of magnesium in a range of from about 0 to about 400 mg;
an amount of molybdenum in a range of from about 0 to about 0.1 mg;
an amount of inositol in a range of from about 0 to about 50 mg; and
an amount of resveratrol in an amount of about 0 to about 5 mg.

2. The composition of claim 1 where the mixture of micronutrient multivitamin and trace elements comprises:
biotin in an amount of about 0.187 mg;
folate in an amount of about 0.25;
iodine in an amount of about 0.0187 mg;
manganese in an amount of about 1.25 mg;
pantothenic acid in an amount of about 12.5 mg;
selenium in an amount of about 0.0875 mg;
vitamin B1 in an amount of about 1.9 mg;
vitamin B12 in an amount of about 0.0075 mg;
vitamin B2 in an amount of about 2.1 mg;
vitamin B3 in an amount of about 25 mg;
vitamin B6 in an amount of about 2.5 mg;
vitamin C in an amount of about 75 mg;
vitamin D in an amount of about 0.0125 mg;
vitamin E in an amount of about 20 mg;
vitamin K in an amount of about 0.056 mg;
lycopene in an amount of about 1.25 mg;
zinc in an amount of about 18.7 mg;
alpha lipoic acid in an amount of about 125 mg;
astaxanthin in an amount of about 0.31 mg;
lutein in an amount of about 6.25 mg;
vitamin A in an amount of about 0.938 mg;
quercetin in an amount of about 64 mg;
glutathione in an amount of about 125 mg; and
where calcium, chromium, copper, magnesium, molybdenum, inositol and resveratrol are not present.

2. The composition of claim 2 where glutathione is replaced with either pine bark extract in an amount of about 20 mg, or curcumin extract in an amount of about 250 mg.

3. The composition of claim 1 further comprising:
calcium in an amount of ≦about 250 mg;
chromium in an amount of ≦about 0.2 mg;
copper in an amount of ≦about 1 mg;
magnesium in an amount of ≦about 100 mg;
molybdenum in an amount of ≦about 0.056 mg;
inositol in an amount of ≦about 50 mg;
pine bark extract in an amount of ≦about 50 mg; and
resveratrol in an amount of ≦about 5 mg;
wherein the amount of quercetin is ≦about 64 mg;
and wherein the amount of glutathione is ≦about 125 mg.

4. The composition of claim 3 where glutathione is replaced with either pine bark extract in an amount of ≦about 20 mg, or curcumin extract in an amount of ≦about 250 mg.

5. The composition of claim 1 where the mixture of micronutrient multivitamin and trace elements comprises:
biotin in an amount of about 0.45 mg;
folate in an amount of about 0.6;
iodine in an amount of about 0.15 mg;
manganese in an amount of about 4 mg;
pantothenic acid in an amount of about 15 mg;
selenium in an amount of about 0.1 mg;
vitamin B1 in an amount of about 2.25 mg;
vitamin B12 in an amount of about 0.009 mg;
vitamin B2 in an amount of about 2.55 mg;
vitamin B3 in an amount of about 30 mg;
vitamin B6 in an amount of about 3 mg;
vitamin C in an amount of about 150 mg;
vitamin D in an amount of about 0.025 mg;
vitamin E in an amount of about 66.7 mg;
vitamin K in an amount of about 0.08 mg;
lycopene in an amount of about 3 mg;
zinc in an amount of about 15 mg;
alpha lipoic acid in an amount of about 100 mg;
astaxanthin in an amount of about 0.25 mg;
lutein in an amount of about 5 mg;
vitamin A in an amount of about 1.5 mg;
quercetin in an amount of about 64 mg;
glutathione in an amount of about 125 mg; and
where calcium, chromium, copper, magnesium, molybdenum, inositol and resveratrol are not present.

6. The composition of claim 5 where glutathione is replaced with either pine bark extract in an amount of about 20 mg, or curcumin extract in an amount of about 250 mg.

7. The composition of claim 5 further comprising:
calcium in an amount of about 250 mg;
chromium in an amount of about 0.2 mg;
copper in an amount of about 1 mg;
magnesium in an amount of about 100 mg;
molybdenum in an amount of about 0.056 mg;
inositol in an amount of about 50 mg;
pine bark extract in an amount of about 50 mg; and
resveratrol in an amount of about 5 mg.

8. The composition of claim 7 where glutathione is replaced with either pine bark extract in an amount of about 20 mg, or curcumin extract in an amount of about 250 mg.

9. The composition of claim 1 where the mixture of micronutrient multivitamin and trace elements comprises:
vitamin A in an amount of about 5,000 IU (1.5 mg);
vitamin C in an amount of about 150 mg;
vitamin D in an amount of about 1,000 IU (0.025 mg);
vitamin E in an amount of about 100 IU (100 mg);
vitamin K in an amount of about 0.080 mg;
vitamin B1 in an amount of about 2.25 mg;
vitamin B2 in an amount of about 2.55 mg;
vitamin B3 in an amount of about 30 mg;
vitamin B6 in an amount of about 3 mg;
folate in an amount of about 0.6 mg;
vitamin B12 in an amount of about 0.009 mg;
biotin in an amount of about 0.45 mg;
calcium in an amount of about 250 mg;
iodine in an amount of about 0.15 mg;
magnesium in an amount of about 100 mg;
zinc in an amount of about 15 mg;
selenium in an amount of about 0.1 mg;
copper in an amount of about 1 mg;
manganese in an amount of about 4 mg;
chromium in an amount of about 0.2 mg;
molybdenum in an amount of about 0.056 mg;
boron in an amount of about 1 mg; and
vanadium in an amount of about 0.05 mg;

10. The composition of claim 9 further comprising:
alpha lipoic acid in an amount of about 100 mg;
citrus bioflavonoids in an amount of about 64 mg;
pine bark extract in an amount of about 50 mg;
resveratrol in an amount of about 5 mg;
lutein in an amount of about 5 mg;
lycopene in an amount of about 3 mg; and
astaxanthin in an amount of about 0.25 mg

11. The composition of claim 10 further comprising:
Resveratrol in an amount of about 5 mg;
N-acetyl cysteine in any amount; and
Beta-carotene in any amount up to the total claim for vitamin A

12. The composition of claim 11 where N-acetyl cysteine replaced with glutathione or curcumin extract in any amount that is generally recognized as safe.

13. A method of treatment for a subject exposed to a radiation source or an oxidative stress, or both, with a radiation-oxidative exposure treatment composition, the method of treatment comprising the steps of:
administering to the subject a daily dose of the radiation-oxidative exposure treatment composition of claim 1
such that the life shortening effects induced by the radiation source or the oxidative stress are ameliorated.

14. The method of claim 13 where the administration of the daily dose of the radiation-oxidative exposure treatment composition occurs on a continuing daily basis for at least 7 days before exposure to the radiation source or oxidative stress.

15. The method of claim 13 where the administration of the daily dose of the radiation-oxidative exposure treatment composition occurs on a continuing daily basis after exposure to the radiation source or oxidative stress.

16. The method of claim 13 further comprising the step of administering to the subject an amount of about 0.004 grams of manganese superoxide dismutase (MnSOD) plasmid DNA in liposome (at a dilution of 100 μg of plasmid DNA per 100 μL of liposomes) per kilogram of the subject's bodyweight at least 24 hours before exposure to the radiation source.

17. The method of claim 13 where the subject is a human being.

18. The method of claim 13 where the daily dose of the radiation-oxidative exposure treatment composition is administered proportionally during the 24-hour period such that the sum of the proportional amounts of the administered radiation-oxidative exposure treatment composition during the 24-hour period totals the daily dose.

19. The method of claim 13 where the daily dose of the radiation-oxidative exposure treatment composition is administered by separately and proportionally administering the daily doses of the micronutrient multivitamin and trace element mixture, the antioxidant and chemopreventative agent mixture, and optionally the fatty acid mixture comprising the radiation-oxidative exposure treatment composition
such that the sum of the proportional amounts of the administered micronutrient multivitamin and trace element mixture during the 24-hour period totals the daily dose of the micronutrient multivitamin and trace element mixture,
such that the sum of the proportional amounts of the administered antioxidant and chemopreventative agent mixture during the 24-hour period totals the daily dose of the antioxidant and chemopreventative agent mixture,
such that the sum of the proportional amounts of the optionally administered fatty acid mixture during the 24-hour period totals the daily dose of the fatty acid mixture, and
such that the sum of the proportional amounts of the administered radiation-oxidative exposure treatment composition during the 24-hour period totals the daily dose of the radiation-oxidative exposure treatment composition.
